(19) United States
(12) Patent Application Publication
(10) Pub. No.: US 2020/0329795 A1
(43) Pub. Date: Oct. 22, 2020

(54) Pocketed Neck Scarf

(71) Applicant: Katherine Rhodes, Ardmore, OK (US)

(72) Inventor: Katherine Rhodes, Ardmore, OK (US)

(21) Appl. No.: 16/387,265

(22) Filed: Apr. 17, 2019

(51) Int. Cl. A41D 23/00; A41D 27/20

(52) U.S. Cl. CPC A41D 23/00; A61F 7/02; A41D 27/20

(57) ABSTRACT

A pocketed neck scarf for easy wrapping and storing pocket warmers includes a scarf body having a bottom side and a top side. The scarf body comprises a base section and a pair of arms extending from the base section. One of the pair of arms is a looped arm and the other arm is a straight arm that is selectively engageable through the looped arm to secure the scarf body around a user's neck with the base section resting on her upper back. The base section has a plurality of pockets on the top side with each pocket configured to receive a pocket warmer.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to neck scarfs and more particularly pertains to a new neck scarf for easy wrapping and storing pocket warmers.

### BRIEF SUMMARY OF THE INVENTION

An embodiment of the disclosure meets the needs presented above by generally comprising a scarf body having a bottom side and a top side. The scarf body comprises a base section and a pair of arms extending from the base section. One of the pair of arms is a looped arm and the other arm is a straight arm that is selectively engageable through the looped arm to secure the scarf body around a users neck with the base section resting on her upper back. The base section has a plurality of pockets on the top side with each pocket configured to receive a pocket warmer.

The disclosure also provides a method for producing a pocketed neck scarf, the method generally comprising obtaining a rectangular piece of material having a right edge, a left edge, an upper edge, and a lower edge. The upper and the lower edges have a height less than a width of the right and left edges. Orient the upper edge to the left such that latitudinal lines run from the upper edge to the lower edge and longitudinal lines run from the right edge to the left edge. A medial, latitudinal cut line is made from the upper edge. The cut line has a length less than the width of the material. The material is folded latitudinally from the cut line to align with each of the right edge and the left edge to form a squared U-shape rotated 90° counterclockwise having a pair of arms extending from a base section. The material is then folded longitudinally to align the lower edge with the end of the cut line, effectively creating a medial fold in the base section to align the lower edge with the pair of arms. Sewing is then stitched along the right edge, the left edge, through the pair of arms adjacent the base section, and through the base section along a pair of pocket stitch lines perpendicularly extending from the medial fold and aligning with a folded inner side of each of the pair of arms. One of the pair of arms is folded longitudinally to align the upper edge within an extension distance left of the base section. The extension distance is greater than zero. The folded upper edge is then sewn to turn the folded arm into a looped arm.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a pocketed neck scarf according to an embodiment of the disclosure.

FIG. 2 is a bottom plan view of an embodiment of the disclosure.

FIG. 3 is a front in-use view of an embodiment of the disclosure.

FIG. 4 is a rear in-use view of an embodiment of the disclosure.

FIG. 5 is a top plan view of an embodiment of the disclosure.

### DETAILED DESCRIPTION OF THE INVENTION

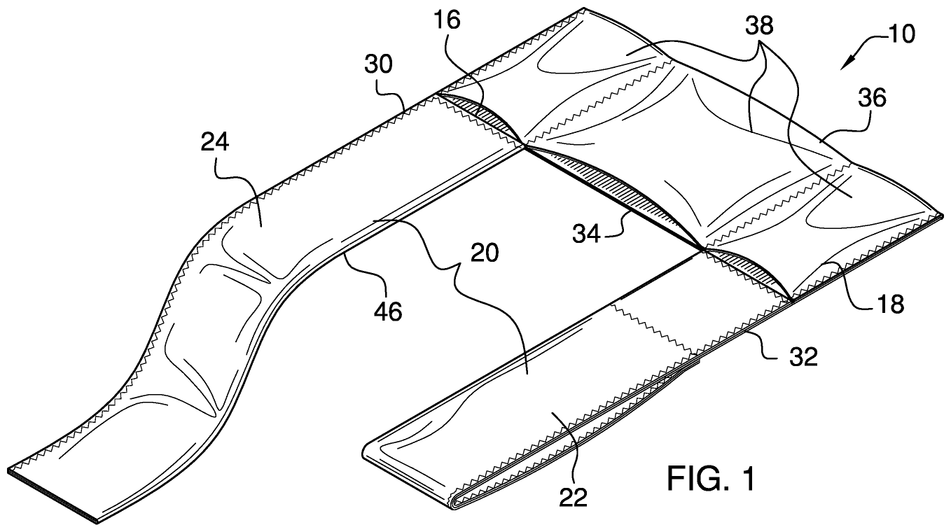

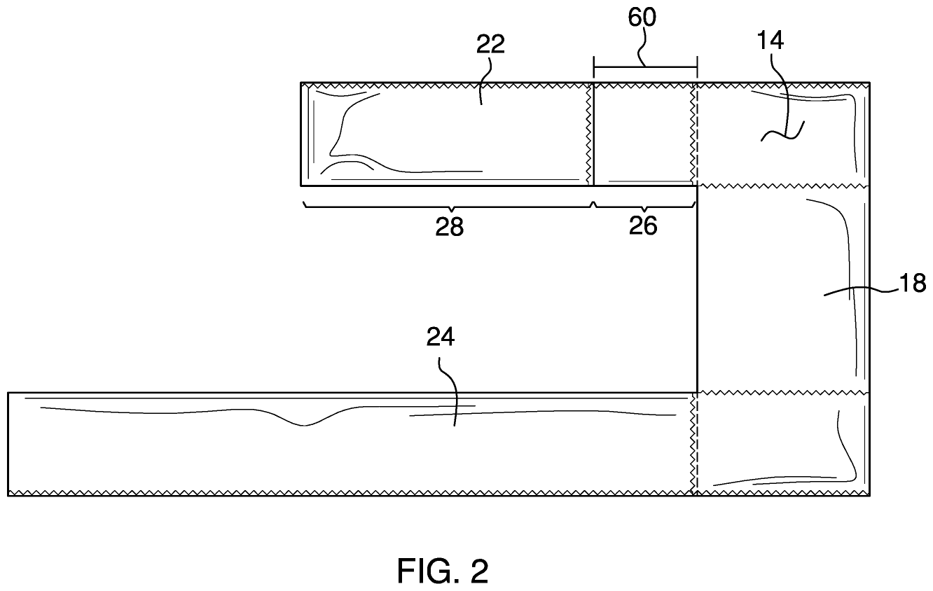

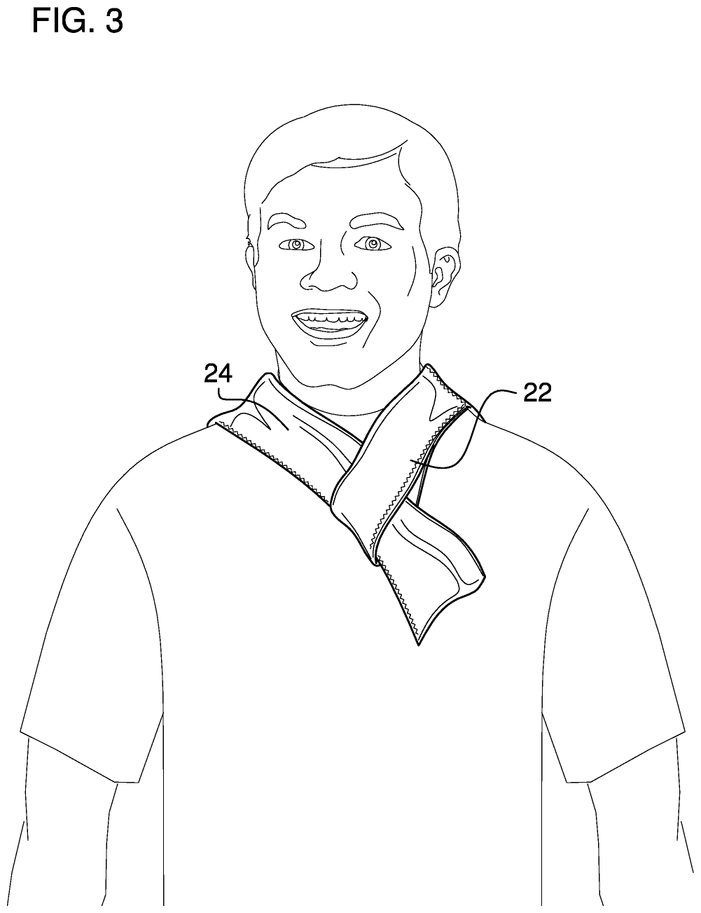

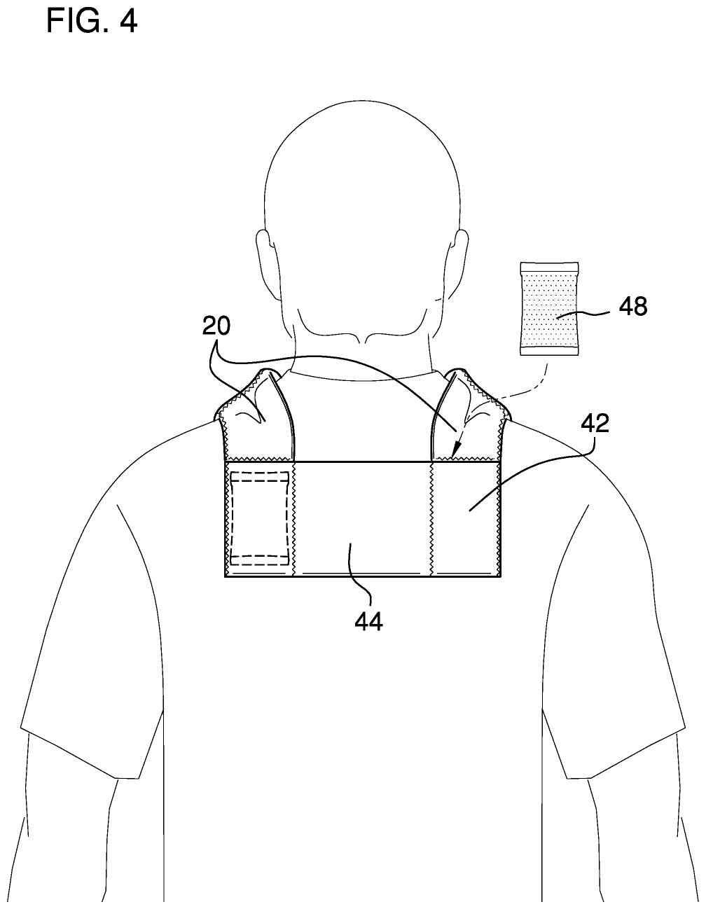

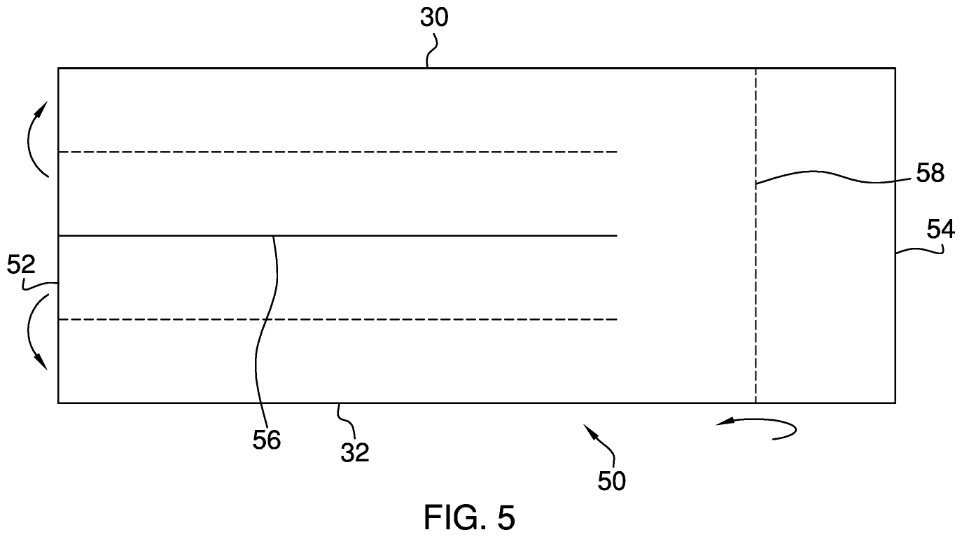

With reference now to the drawings, and in particular to FIGS. 1 through 5 thereof, a new neck scarf embodying the principles and concepts of an embodiment of the disclosure and generally designated by the reference numeral 10 will be described.

As best illustrated in FIGS. 1 through 5, the pocketed neck scarf 10 generally comprises a scarf body 12 having a bottom side 14 and a top side 16. The scarf body 12 comprises a base section 18 and a pair of arms 20 extending from the base section 18. One of the pair of arms is a looped arm 22 and the other arm is a straight arm 24. The looped arm 22 comprises an extension 26 extending from the base section 18 and a looped portion 28 adjacent the extension 26. The length of the straight arm 24 is equal to the sum of two times the length of the looped portion 28 and the length of the extension 26. The straight arm 24 is selectively engageable through the looped arm 22 to secure the scarf body 12 around a user's neck with the base section 18 resting on her upper back.

The base section 18 is rectangular and has a right edge 30, a left edge 32, a top edge 34, and a bottom edge 36. The pair of arms 20 is aligned with the right edge 30 and the left edge 32. The bottom side 14 and the top side 16 may be a single piece of material folded along the bottom edge 36 and stitched along each of the right edge 30 and the left edge 32. The base section 18 has a plurality of pockets 38 on the top side 16. The plurality of pockets 38 may be defined by a pair of pocket stitch lines 40 perpendicularly extending from the bottom edge 36 to the top edge 34 with each pocket 16 open along the top edge 34. The plurality of pockets 38 thus comprises a pair of outer pockets 42 and a medial pocket 44. The pair of pocket stitch lines 40 aligns with an inner side 46 of the pair of arms 20. The pair of outer pockets 42 and the pair of arms 20 thus have an equal width. Each of the plurality of pockets 38 is configured to receive a pocket warmer 48.

In use, pocket warmers 48 are activated and placed within the plurality of pockets 38. The base section 18 is then worn on the upper back with the pair of arms 20 draping over the user's shoulders. The straight arm 24 is placed through the looped portion 28 of the looped arm 22 to secure the scarf body 12 and to cover the front of the user's neck.

The scarf body 12 may be produced from a single rectangular piece of material 50 having the right edge 30, the left edge 32, an upper edge 52, and a lower edge 54. The upper 52 and the lower edges 54 have a height less than a width of the right 30 and left edges 32. The height is 40% of the width to create ideal proportions for design practicality. Orient the upper edge 52 to the left such that latitudinal lines run from the upper edge 52 to the lower edge 54 and longitudinal lines run from the right edge 30 to the left edge 32. Make a medial, latitudinal cut line 56 from the upper edge 52. The cut line 56 has a length less than the width of the material 50 and may be two thirds the width to create the proper sized base portion 18 and length of the pair of arms 20. Fold the material 50 latitudinally from the cut line 56 to align with each of the right edge 30 and the left edge 32. The material 50 is now a squared U-shape rotated 90° counterclockwise having the pair of arms 20 extending from the base section 18. Fold the material 50 longitudinally to align the lower edge 54 with the end of the cut line 56, effectively creating a medial fold 58 in the base section 18 to align the lower edge 54 with the pair of arms 20. The medial fold 58 becomes the bottom edge 36 of the base section and the lower edge 54 becomes the top edge 34 of the base section. Sew along the right edge 30, the left edge 32, through the pair of arms 20 adjacent the base section 18, and through the base section 18 along the pair of pocket stitch lines 40 aligning with the folded inner side 46 of each of the pair of arms. Fold one of the pair of arms 20 longitudinally to align the upper edge 52 within an extension distance 60 left of the base section 18. Sew along the folded upper edge 54 to turn the folded arm into the looped arm 22. The extension distance 60 is greater than zero and may be less than 10% of the width of the material to create a proper difference between the length of the straight arm 24 and the overall length of the looped arm 22.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A pocketed neck scarf comprising:
a scarf body having a bottom side and a top side, the scarf body comprising a base section and a pair of arms extending from the base section, one of the pair of arms being a looped arm and the other arm being a straight arm, the straight arm being selectively engageable through the looped arm to secure the scarf body around a user's neck with the base section resting on her upper back; and
the base section having a plurality of pockets on the top side, each of the plurality of pockets being configured to receive a pocket warmer.

2. The pocketed neck scarf of claim 1 further comprising the base section being rectangular and having a right edge, a left edge, a top edge, and a bottom edge, the pair of arms being aligned with the right edge and the left edge and the plurality of pockets being open along the top edge.

3. The pocketed neck scarf of claim 2 wherein the bottom side and the top side are a single piece of material folded along the bottom edge and stitched along each of the right edge and the left edge, the plurality of pockets being defined by a pair of pocket stitch lines perpendicularly extending from the bottom edge to the top edge and thus comprising a pair of outer pockets and a medial pocket.

4. The pocketed neck scarf of claim 3 wherein the pair of pocket stich lines align with an inner side of the pair of arms, the pair of outer pockets and the pair of arms thus having an equal width.

5. The pocketed neck scarf of claim 1 further comprising the looped arm being between one half and two thirds the length of the straight arm.

6. The pocketed neck scarf of claim 1 further comprising the looped arm comprising an extension extending from the base section and a looped portion adjacent the extension, the extension having a length less than 30% of the overall length of the looped arm.

7. The pocketed neck scarf of claim 6 further comprising the length of the straight arm being equal to the sum of two times the length of the looped portion and the length of the extension.

8. The pocketed neck scarf of claim 1 further comprising the scarf body being constructed from a single rectangular piece of material.

9. A pocketed neck scarf comprising:
a scarf body having a bottom side and a top side, the scarf body comprising a base section and a pair of arms extending from the base section, one of the pair of arms being a looped arm and the other arm being a straight arm, the looped arm comprising an extension extending from the base section and a looped portion adjacent the extension, the length of the straight arm being equal to the sum of two times the length of the looped portion and the length of the extension, the straight arm being selectively engageable through the looped arm to secure the scarf body around a user's neck with the base section resting on her upper back;
the base section being rectangular and having a right edge, a left edge, a top edge, and a bottom edge, the pair of arms being aligned with the right edge and the left edge, the bottom side and the top side being a single piece of material folded along the bottom edge and stitched along each of the right edge and the left edge; and
the base section having a plurality of pockets on the top side, the plurality of pockets being defined by a pair of pocket stitch lines perpendicularly extending from the bottom edge to the top edge and being open along the top edge, thus comprising a pair of outer pockets and a medial pocket, the pair of pocket stitch lines aligning with an inner side of the pair of arms, the pair of outer pockets and the pair of arms thus having an equal width, each of the plurality of pockets being configured to receive a pocket warmer.

10. A method for producing a pocketed neck scarf comprising:
obtaining a rectangular piece of material having a right edge, a left edge, an upper edge, and a lower edge, the upper and the lower edges having a height less than a width of the right and left edges;
orienting the upper edge to the left such that latitudinal lines run from the upper edge to the lower edge and longitudinal lines run from the right edge to the left edge;
making a medial, latitudinal cut line from the upper edge, the cut line having a length less than the width of the material;
folding the material latitudinally from the cut line to align with each of the right edge and the left edge, the material now being a squared U-shape rotated 90° counterclockwise having a pair of arms extending from a base section;
folding the material longitudinally to align the lower edge with the end of the cut line, effectively creating a medial fold in the base section to align the lower edge with the pair of arms;
sewing along the right edge, the left edge, through the pair of arms adjacent the base section, and through the base section along a pair of pocket stitch lines perpendicularly extending from the medial fold and aligning with a folded inner side of each of the pair of arms;
folding one of the pair of arms longitudinally to align the upper edge within an extension distance left of the base section, the extension distance being greater than zero; and
sewing along the folded upper edge to turn the folded arm into a looped arm.

11. The method for producing a pocketed neck scarf of claim 10 further comprising the height being 40% of the width.

12. The method for producing a pocketed neck scarf of claim 10 further comprising the length of the cutline being two thirds the width of the material.

13. The method for producing a pocketed neck scarf of claim 10 further comprising the extension distance being less than 10% of the width of the material.
